(19) United States
(12) Patent Application Publication
(10) Pub. No.: US 2023/0126374 A1
(43) Pub. Date: Apr. 27, 2023

(54) SYSTEM, METHOD, AND COMPUTER PROGRAM FOR ASYNCHRONOUS AND SYNCHRONOUS MEDICAL INTERACTIONS BASED ON DATA MINING

(71) Applicant: Legrande Corporation, Rexburg, ID (US)

(72) Inventors: Anthony Sine, Laguna Beach, CA (US); Malaquis Alex Lopez, Fullerton, CA (US)

(21) Appl. No.: 17/510,192

(22) Filed: Oct. 25, 2021

(51) Int. Cl. G16H 10/60

(52) U.S. Cl. CPC G16H 10/60

(57) ABSTRACT

A system, method, and non-transitory computer readable storage medium are provided for asynchronous and synchronous medical interactions based on data mining. In use, asynchronous medical data associated with a patient is stored, and synchronous medical data associated with the patient is stored. The asynchronous medical data and the synchronous medical data are integrated to create cohesive patient data. Additionally, relevant data is extracted from clinical databases based on the cohesive patient data. Further, one or more decisions are provided based on the cohesive patient and the relevant data.

### RELATED APPLICATIONS

The present application incorporates by reference the following application by reference in their entirety for all purposes: U.S. application Ser. No. 17/510,166 entitled “SYSTEM, METHOD, AND COMPUTER PROGRAM FOR RECOMMENDED MEDICAL TREATMENTS BASED ON DATA MINING” filed Oct. 25, 2021 (docket number LEG1P001).

### FIELD OF THE INVENTION

The present invention relates to medical interactions, and more particularly to asynchronous and synchronous medical interactions based on data mining.

### BACKGROUND

Currently, the medical industry lacks accurate feedback between instructions (or prescriptions given) by a medical provider, and compliance or completion of such instructions. Patients may choose not to fulfill a prescription, may have personal preferences or biases which may render such prescription not viable, and/or may commence in part the prescription but not timely complete it. Medical providers therefore often do not receive any feedback on patient action(s) after they leave the office of the medical provider.

As such, there is thus a need for addressing these and/or other issues associated with the prior art.

### SUMMARY

A system, method, and non-transitory computer readable storage medium are provided for asynchronous and synchronous medical interactions based on data mining. In use, asynchronous medical data associated with a patient is stored, and synchronous medical data associated with the patient is stored. The asynchronous medical data and the synchronous medical data are integrated to create cohesive patient data. Additionally, relevant data is extracted from clinical databases based on the cohesive patient data. Further, one or more decisions are provided based on the cohesive patient and the relevant data.

### BRIEF DESCRIPTION OF THE DRAWINGS

FIG. 1 illustrates a method for providing one or more decisions based on data mining, in accordance with one embodiment.

FIG. 2 illustrates an online network architecture for asynchronous and synchronous medical interactions, in accordance with one embodiment.

FIG. 3 illustrates a system for patient interactions, in accordance with one embodiment.

FIG. 4 illustrates a method for receiving feedback from a patient, in accordance with one embodiment.

FIG. 5 illustrates user interfaces for interacting with a patient, in accordance with one embodiment.

FIG. 6 illustrates a method for tracking patient compliance and refill management, in accordance with one embodiment.

FIG. 7 illustrates a system for doctor-led interaction, in accordance with one embodiment.

FIG. 8 illustrates system for patient-led communication, in accordance with one embodiment.

FIG. 9 illustrates a method for providing an omni-channel support and medical delivery system, in accordance with one embodiment.

FIG. 10 illustrates a method for updating and sending recommendations to patient, in accordance with one embodiment.

FIG. 11 illustrates a network architecture, in accordance with one possible embodiment.

FIG. 12 illustrates an exemplary system, in accordance with one embodiment.

FIG. 13 illustrates an exemplary therapeutic interchange, in accordance with one embodiment.

### DETAILED DESCRIPTION

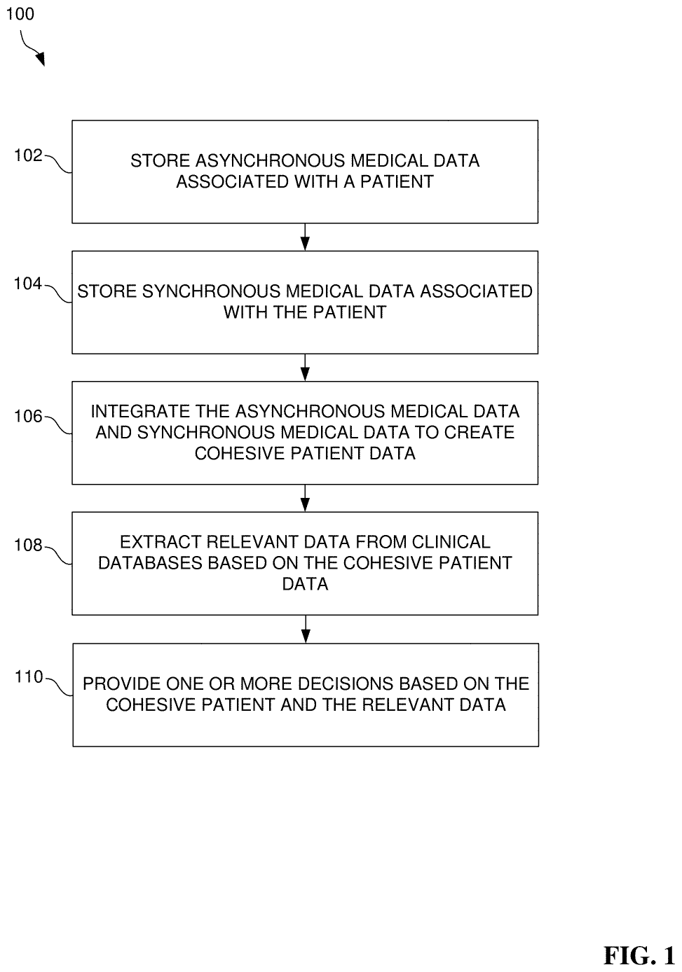

FIG. 1 illustrates a method 100 for providing one or more decisions based on data mining, in accordance with one embodiment.

As shown, asynchronous medical data associated with a patient is stored. See operation 102. Within the context of the present description, asynchronous medical data may include any non real-time data. For example, asynchronous medical data may include patient medical history, transmission of content (including but not limited to text, email, voice message) retrieved on demand, lifestyle habits (including but not limited to diet, exercise, tobacco use, alcohol use, etc.), personal data (including but not limited to sex, date of birth, ethnicity, age of when condition was diagnosed if applicable, etc.), remote patient monitoring (based on collected and/or transmitted data), at least one patient action, and/or any other medically related data or medically related action.

In one embodiment, patient medical history may include one or more of: patient and medical provider interaction, patient medical procedure(s), patient medical condition(s), patient medical allergy(ies), family medical procedure(s), family medical condition(s), or family medical allergy(ies), prior medical interaction, diagnosis, prescription(s), illness(es), surgery(ies), immunization(s), results of physical exams and tests, family health history, frequency of past screening, genetic test(s), substance abuse, pregnancy complications, past hospitalizations, etc.

Additionally, the patient action may include one or more of: receipt of medical treatment, refill of a prescription, decision to not take medical action, refusal of medical treatment, election of an alternative treatment, selection of a lower-cost option, response to or sending a communication from/to a medical representative, a change a health insurance plan, fulfillment of medical prescription, completion of medical prescription, tracking of dosage, a request for refill, indication of drug side effect, completion of daily reminder relating to medical prescription, completion of interaction with caregiver or medical provider, tracking of exercise, tracking of diet, tracking of food intake, body vital measurements, body vital statistics, body vital historical data, and/or fulfillment of medical prescription coupon.

In one embodiment, the asynchronous medical data may be stored on a server (e.g. cloud or server-based electronic medical record system, dedicated medical server, HIPAA-compliant data storage, etc.). In another embodiment, the asynchronous medical data may be stored locally (e.g. on a patient's device, etc.) and then uploaded to a server at a later time. Additionally, data may be synced between a patient's device and a server (including but not limited to two-way sync, one-way sync, etc.). Such syncing of data may be based on privacy limitations, release of medical records, age of patient (or age of minor patient, etc.), healthcare release of information, etc.

Additionally, synchronous medical data associated with the patient is stored. See operation 104. Within the context of the present description, synchronous medical data may include any real-time data. For example, synchronous medical data may include real-time behavior observations, real-time communication, digital communication, video patient evaluations, health triage, in-person visit/communication (face-to-face communication), live examination, telehealth communication, in-house provider visit, in-office provider visit, current patient and/or vital information (height, weight, symptom(s), temperature, pain, blood pressure, heart rate, etc.), etc. In one embodiment, synchronous medical data may be obtained in-person. In other embodiments, synchronous medical data may be obtained remotely (e.g. via telehealth solutions, video chat, phone call, etc.). The synchronous medical data may be based also on one or more sensing devices (e.g. smart watch, remote vitals monitoring (e.g. body temperature, heart rate, RR interval, respiration rate, oxygen level, etc.), wearables, ambient sensors (e.g. movement, fall detection, sleep cycles, etc.), etc.).

As used herein, the term asynchronous medical data may be synonymous with asynchronous data within the context of the present description, and the term synchronous medical data may be synonymous with synchronous data within the context of the present description.

Further, the asynchronous medical data and synchronous medical data are integrated to create cohesive patient data. See operation 106. In one embodiment, cohesive patient data may include retaining separate data sets for each of the asynchronous medical data and the synchronous medical data. In another embodiment, the cohesive patient may combine all of the asynchronous medical data and the synchronous medical data into one global data set. Additionally, metadata associated with each of the asynchronous data and the synchronous data may either be maintained (such that the source of each data may be verified) within the cohesive patient data, and/or the metadata may be updated (or inserted if missing) for each data of the asynchronous data or the synchronous data. For example, the metadata may be updated with information relating to a data of update, the reason for the creation of cohesive patient data (for example, the data is being combined at the request of doctor XX on date XX for evaluation, etc.), the location of the update, what specific data of the asynchronous data and/or synchronous data is being retrieved, etc.

In addition, relevant data is extracted from clinical databases based on the cohesive patient data. See operation 108. Within the context of the present description, clinical databases may include (but not be limited to) clinical study results, multi-institution clinic databases for research, physician database, clinic management system data, hospital patient database, single practice clinical database (e.g. such from a single doctor or practice of doctors, etc.), research database (e.g. PubMed, EMBASE, Cochrane Library, PubMed Central, and/or UpToDate, etc.), and/or any other collection of organized clinical/patient data.

Additionally, in another embodiment, the clinic databases may include one or more patient groups where the one or more patient groups may be based on at least one of: age, weight, race, medical condition, medical provider, medical insurance, or location. Further, in one embodiment, the relevant data may include medical data, and/or any other data which may be relevant to the patient.

In one embodiment, the cohesive patient data may be anonymized prior to being used as a basis for extracting the relevant data. For example, identifiable and/or traceable links, information, and/or data may be removed from the cohesive patient data. Additionally, using the cohesive patient data may be further based on receiving one or more permissions from the patient (e.g. granting authorization, granting in-part authorization, allowing the data to be used in an anonymous manner, etc.). In this manner, the use of the cohesive patient data may comply with legal (e.g. HIPAA, etc.), regulatory, and/or medical legal privacy requirements prior to be used to extract the medical data. Additionally, use of the cohesive patient data (e.g. open-access versus controlled-access model) may dictate the level of anonymization required before being used as a basis for extracting the relevant data.

Further, one or more decisions are provided based on the cohesive patient and the relevant data. See operation 110. For example, the one or more decisions may include a clinical practice guideline, one or more medical actions (e.g. to receive treatment, cancer screening, follow up care, etc.), lifestyle change (e.g. get more sleep, eat less sugar, smoke less, get more exercise, etc.), a prescription (e.g. drug, medicine, etc.), etc. In this manner, the one or more decisions may be based on a history of the patient, a current state of the patient, and data associated with others with similar history and/or current state.

Furthermore, the one or more decisions may be further refined based on additional input from physician. For example, after reviewing the one or more decisions, the physician may provide additional input based on historical interactions of the patient, pricing considerations (e.g., insurance co-payments, generic, etc.), lifestyle considerations (e.g. vegetarian, religious constraint, etc.), and availability considerations (e.g. location of patient, stocking at preferred pharmacy, etc.). U.S. application Ser. No. 17/510,166 entitled “SYSTEM, METHOD, AND COMPUTER PROGRAM FOR RECOMMENDED MEDICAL TREATMENTS BASED ON DATA MINING” filed Oct. 25, 2021 (docket number LEG1P001) discloses in further detail the use of attributes associated with a patient which can be used to filter down the one of more recommendations, the content of such application of which is incorporated herein by reference for all purposes.

In various embodiments, the method 100 may be implemented in a variety of scenarios. For example, doctors are often blind as to what occurs with their patients after they leave the doctor's office, and thus, are unable to personally tailor the patient's recovery efforts (beyond giving a one-time diagnosis). The doctor, therefore, would benefit from additional information that the patient takes. This additional information may be the asynchronous medical data, as described herein (see, e.g., operation 102). Within the context described hereinabove, one scenario for operation 104 would be a first real-time encounter with a doctor relating to a particular set of symptoms. In addition to a first real-time encounter, operation 104 may also be construed as any follow-up real-time interaction with the patient. Under this context, therefore, operation 102 may be construed as including prior medical information (prior to the diagnosis) but may include additional medical information which is provided after the diagnosis, such that any follow-up interaction between the doctor and the patient can be based on additional information. This additional medical information can be integrated with additional real-time data (in a manner consistent with operation 106) to provide a new set of cohesive patient data, which in turn can be then the basis for extracting relevant data (in a manner consistent with operation 108), and an updated set of recommendations (in a manner consistent with operation 110) may be provided. As such, the method 100 may include a variety of scenarios, from a first-time diagnosis of symptoms to any level of follow-up care.

In one embodiment, the concept of data mining, as it relates to the method 100, covers analyzing data sets and medical data sets, and as described hereinabove, the medical data sets may include any or any combination of asynchronous data, synchronous data, patient relevant data, and/or medical data from clinical databases.

The asynchronous medical data (based on operation 102) may provide a doctor with the ability to understand what the patient is doing (after diagnosis) such that the doctor can more effectively then manage the interactions outside of the office and remain in control of the recovery of the patient. In this manner, the method 100 may relate to after care instructions and actions, such that actions of the patient (e.g. what product the patient purchased, whether the patient did what was asked, whether the patient used the product recommended, whether the patient made lifestyle changes discussed, etc.) can be provided back to the doctor for more personalized follow up care. In this manner, the method 100 may be viewed additional as being a cycle such that when operations 102-110 are completed, the cycle may start over again (operation 110 continues on operation 102) whenever the asynchronous medical data has been updated in some manner. In one embodiment, the method 100 may be implemented without fulfilling all operations 102-110. For example, after initial diagnosis, the doctor may be able to care for the patient based on the updated asynchronous medical data and the relevant data from clinical databases, such that synchronous medical data (based on real-time interaction) may not be required. Each patient may require a separate, different, and personalized level of follow-up care. As such, the method 100 may be adapted as needed to best manage and care for patients after diagnosis.

In one embodiment, the method 100 may repeat (for any number of iterations) with the synchronous medial data being optional. The doctor may direct, however, when synchronous medical data (i.e. live exam) is necessary in order to more accurately assist the patient with recovery. As such, in some instances, a live exam may be required upfront (as part of initial diagnosis), or at any particular time during the course of recovery, which may be dictated by the doctor, and based contextually on the diagnosis of the issue (e.g. a higher risk diagnosis may require a higher frequency of follow up live exam visits).

As an example, a patient may be suffering from farsightedness (e.g. presbyopia, etc.). The patient may have an exam with an eye doctor to diagnose the issue, whereupon a prescription may be given. As part of the one or more decisions provided by the doctor (in a manner consistent with operation 110), the doctor may recommend eye drops to alleviate the problem. The patient may then take the eyedrops as prescribed by the doctor. The doctor may follow up (e.g. via SMS messaging, via email, etc.) to determine if the patient is taking the eyedrop as prescribed. If the patient has adhered to the prescribed course of action, the doctor may authorize a second prescription of eyedrops to be automatically sent to the patient as soon as the first prescription of eyedrops is completed (e.g. based on feedback from the patient, based on rate of daily use of drops, etc.). In this manner, patient adherence can be tracked, the data can be sent to the doctor for review, and the doctor can dictate whether additional care and prescriptions are needed to continue to remedy the issue.

More illustrative information will now be set forth regarding various optional architectures and uses in which the foregoing method may or may not be implemented, per the desires of the user. It should be strongly noted that the following information is set forth for illustrative purposes and should not be construed as limiting in any manner. Any of the following features may be optionally incorporated with or without the exclusion of other features described.

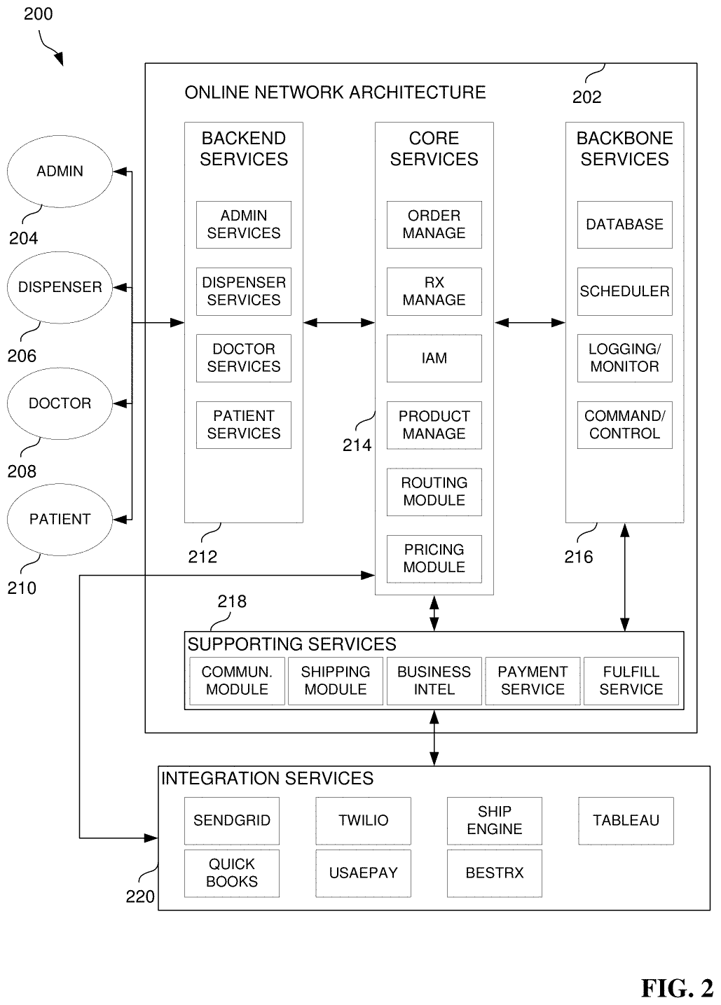

FIG. 2 illustrates an online network architecture 200 for asynchronous and synchronous medical interactions, in accordance with one embodiment. As an option, the online network architecture 200 may be implemented in the context of any one or more of the embodiments set forth in any previous and/or subsequent figure(s) and/or description thereof. Of course, however, the online network architecture 200 may be implemented in the context of any desired environment. Further, the aforementioned definitions may equally apply to the description below.

As shown, the online network architecture 200 is connected (individually and/or collectively, as needed) to an admin 204, a dispenser 206, a doctor 208, and/or a patient 210. In various embodiments, the admin 204 may assist with managing and/or tracking patient medical interactions, the dispenser 206 may assist with providing resources (including prescriptions) based on the recommendation of the doctor 208, and the doctor 208 may assist with providing the medical interaction with the patient 210. Any or all of the admin 204, the dispenser 206, the doctor 208, and/or the patient 210 may interact with the online network architecture 202.

The online network architecture 202 may include backend services 212, core services 214, backbone services 216, and supporting services 218. The backend services 212 may include admin services, dispenser services, doctor services, patient services, and/or any other service which may facilitate front-end delivery of data and/or services. For example, in one embodiment, a user interface may exist for each of the admin 204, the dispenser 206, the doctor 208, and/or the patient 210 as provided by the backend services 212 for frontend interaction. Additionally, each of the services within the backend services 212 may interact. For example, in response to input being received via the doctor services, the input may be sent (at least in part) to the dispenser services to manage fulfillment of a recommendation provided by the doctor 208.

Additionally, the core services 214 may include order management, prescription management, IAM management (including identity access management), product management, routing module, pricing module, and/or any service that relates to that which is essential for the medical interaction. For example, the IAM management may provide identify services for the patient services of the backend services 212. Additionally, in response to input received by the doctor services of the backend services 212, order management and/or prescription management of the core services 214 may be invoked to assist with managing and tracking a prescription associated with the patient 210. Further, the core services 214 may interact with the supporting services 218 and/or the integration services 220 as needed.

The backbone services 216 may include one or more database(s), a scheduler, logging and monitoring, command and control, as well as any other infrastructure necessary for any of the core services 214, the backend services 212, and/or the supporting services 218. In one embodiment, the backbone services 216 include an interconnect (or a path to connect) between one or more other services. For example, order management of the core services 214 may use the scheduler of the backbone services 216 to schedule delivery of an order. In like manner, the other services of the backbone services 216 may provide functionality to enable completion of one or more other services.

The supporting services 218 may include a communication module, a shipping module, a business intelligence module, a payment service, a fulfillment service, and/or any other service necessary to enable any of the backend services 212, the core services 214, and/or the backbone services 216. For example, in one embodiment, when an order is received via the order management of the core services 214, the scheduler of the backbone services 216 may be invoked (as discussed herein), and the shipping module of the supporting services 218 may be invoked, as well as any further service of the integration services 220 (such as the ShipEngine service to line up shipping of the order). As another example, the business intelligence service of the supporting services 218 may provide enterprise processes to integrate and manage various services (such as, in one embodiment, the scheduler of the backbone services 216, the financial services such as QuickBooks of the integration services 220, data visualization services such as tableau of the integration services 220, and/or the database of the backbone services 216). Of course, any specific service of the supporting services may be connected to various components of any of the backend services 212, the core services 214, the backbone services 216, and/or the integration services 220.

The integration services 220 may include a variety of tasks including financial services, shipping services, revenue services, data management, data extraction, data visualization, etc. In various embodiments, the integration services 220 may include SendGrid, Twilio, ShipEngine, Tableau, QuickBooks, USAEPay, and/or BestRX. Of course, any other integration service may be included within the integration services 220 based on the demands and needs of the online network architecture 202.

In various embodiments, the online network architecture 202 may be flexible such that the platform can be adjusted to meet and evolve with a change of business model or a market need. Additionally, the online network architecture 2020 may provide an intuitive user experience such that use of the system may be simple to learn (from a management point of view of all stakeholders), and patients can engage with the system in a logical manner, which in turn, can increase customer retention.

Further, in one embodiment, the online network architecture 202 may be agile such that each part of the system may be managed and changed independently. This aspect may assist with reducing cost to develop, and with improving a speed to market in providing services. Additionally, given that each service is managed independently, any risk or issue may be isolated to a specific service, thereby increasing run time for all other services.

Still yet, in another embodiment, the online network architecture 202 allows for integration of data such that the system can connect to multiple applications simultaneously (e.g. such as through API links, etc.). Further the data may be pulled at any time for analytics and machine learning.

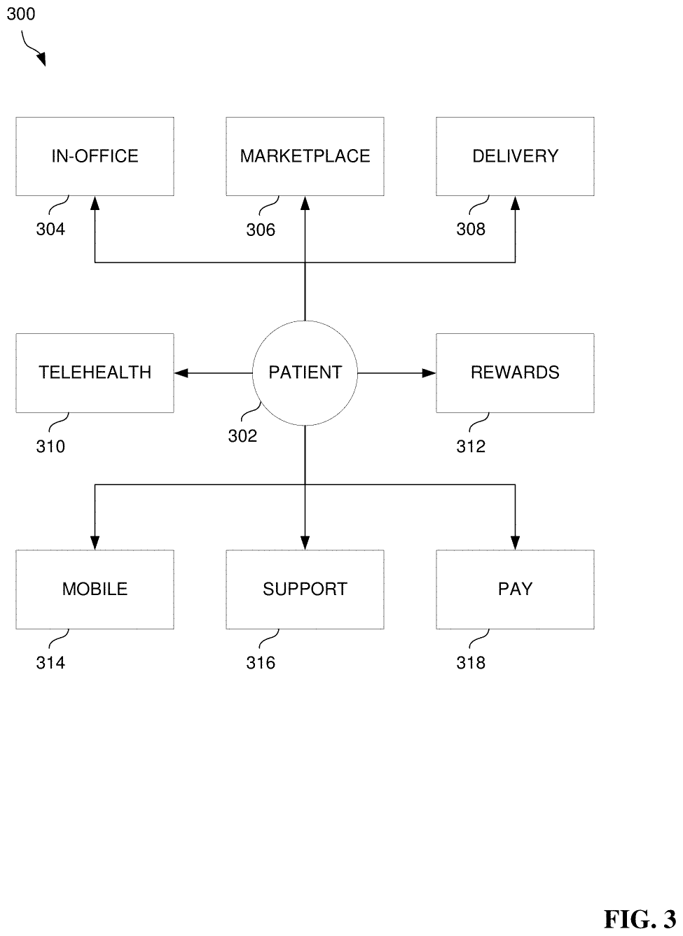

FIG. 3 illustrates a system 300 for patient interactions, in accordance with one embodiment. As an option, the system 300 may be implemented in the context of any one or more of the embodiments set forth in any previous and/or subsequent figure(s) and/or description thereof. Of course, however, the system 300 may be implemented in the context of any desired environment. Further, the aforementioned definitions may equally apply to the description below.

As shown, a patient 302 remains in the center of all interactions. The patient 302 may interact via in-office 304 (e.g. in person medical interaction), medical insurance marketplace 306, delivery 308 (e.g. of prescriptions, of medically-related recommendation, etc.), rewards 312 (e.g. Medicare rewards, completion of healthy activities, completion of financial incentives, etc.), payment 318 (e.g. to pay for medically related bill, to automate medical subscription service, to automate interaction with insurance carrier for processing of bills, etc.), support 316 (e.g. with a medical service provider, etc.), mobile 314 (e.g. mobile delivery service, mobile medical services, etc.), and/or telehealth 310 (e.g. online medical interaction, etc.).

Any or all of the interactions of the system 300 may be tracked as desired and set by the patient 302. For example, the patient 302 may choose to interact with a medical provider via an in-office 304 interaction, but choose not to interact via telehealth 310. Additionally, data associated with a medical interaction may be tracked as desired and set by the patient 302. In this manner, privacy and personal settings may be associated with the interaction and with data resulting from the interaction.

Further, data may exist or be created in relation to any or all of in-office 304, medical insurance marketplace 306, delivery 308, rewards 312, payment 318, support 316, mobile 314, and/or telehealth 310. Such data may be controlled by the patient 302 such that the patient 302 remains at the center of all interactions, all data, and all privacy/security associated with the data. In view of such, the system 300 shows a patient-focused, patient-led, and patient-controlled series of interactions that remain under the direction of the patient 302.

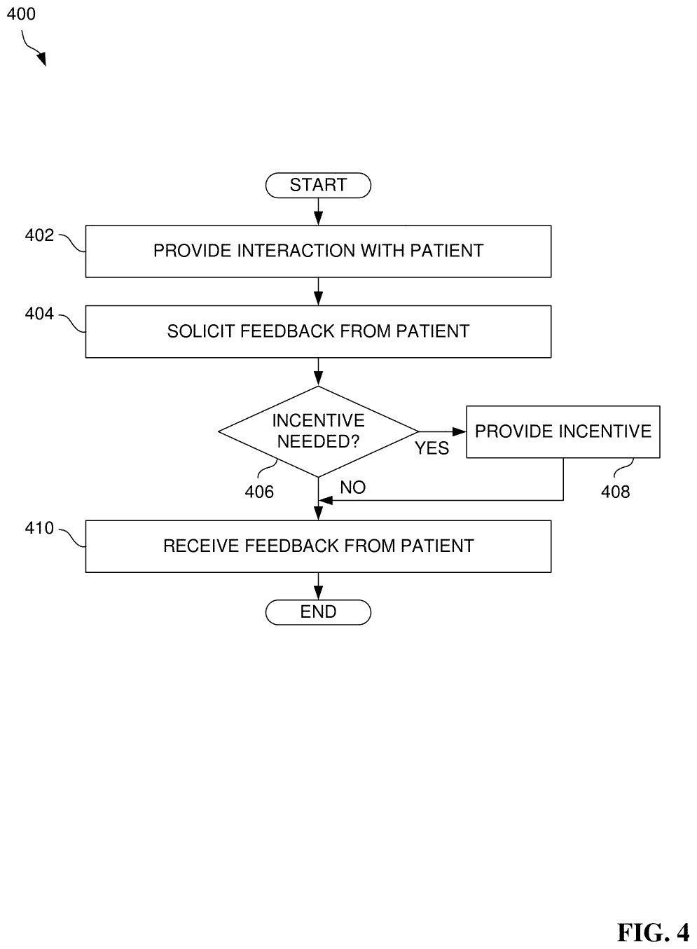

FIG. 4 illustrates a method 400 for receiving feedback from a patient, in accordance with one embodiment. As an option, the method 400 may be implemented in the context of any one or more of the embodiments set forth in any previous and/or subsequent figure(s) and/or description thereof. Of course, however, the method 400 may be implemented in the context of any desired environment. Further, the aforementioned definitions may equally apply to the description below.

As shown, the method 400 starts with providing interaction with a patient. See operation 402. In one embodiment, the interaction may include any medically related interaction with a patient. For example, the medically related interaction may include an in-person visit, a digital interaction (e.g. telehealth, etc.), picking up a prescription, and/or any action by a patient in relation to a medical recommendation. In other words, the interaction of the patient may be in response to either the patient commencing the interaction (e.g. reaching out to the medical provider for services, etc.), or a medical provider reaching out to the patient in any manner.

Feedback may be solicited from the patient. See operation 404. For example, feedback may include any information derived in relation to the patient in response to the interaction of operation 402. For example, in one embodiment, as a result of an in-person medical interaction, a medical provider may prescribe to the patient to take a specific drug. Feedback may be solicited in that the medical provider may wish to know whether the patient is taking the prescription as recommended by the provider, whether the medicine is causing any reaction (e.g. allergic reaction, etc.) with the patient, whether the prescription has resolved the diagnosed issue, etc.

The solicitation of feedback of operation 404 may include a request for information. For example, the request may be in the form of an email, a text message, an app user interface display, etc. Additionally, the request may occur in response to every new interaction (of the interaction of operation 402), or it may be configured once by a user for the present and future medical interactions. For example, the patient may opt for and give permission for a smart device to collect vital statistics (heart rate, blood pressure, etc.) and to relay such information back to the medical provider. In other embodiments, the feedback may relate only to the present interaction (of operation 402), in which case the feedback being solicited (of operation 404) may be on a per-interaction basis (i.e. the feedback may not be set once as it would only relate to the present interaction).

In another embodiment, the patient may desire not to provide any feedback (in any form) to the medical provider, and via operation 404, the patient may set such settings and permissions.

It is determined whether an incentive is needed, per decision 406, where if an incentive is needed, an incentive is provided, per operation 408. In various embodiments, an incentive may include a reminder, an alternative form of reaching out (e.g. by email, by phone, by text message, by hardcopy, etc.), a shorter version of the request for feedback, a financial reward (e.g. “fill out the survey and you'll be entered to win a gift card”, reward miles that can be transferred to an airline or dining card, etc.), send personalized card, send thank-you for visit, etc.

Next, feedback is received form the patient. See operation 410. Such feedback may be used as asynchronous data (per operation 102) for future interactions with a medical provider.

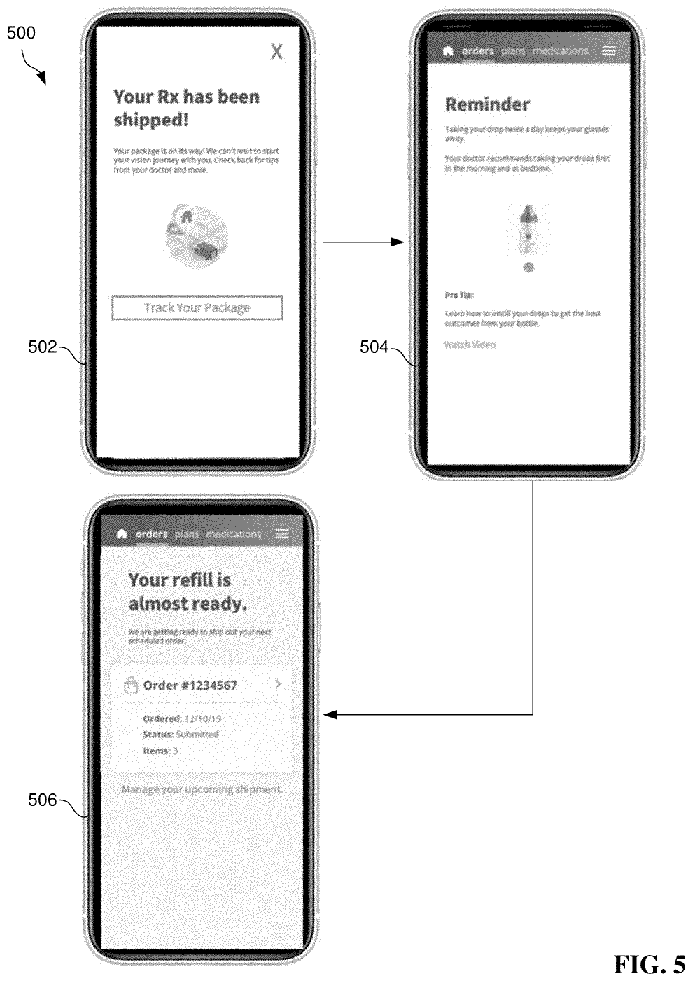

FIG. 5 illustrates user interfaces 500 for interacting with a patient, in accordance with one embodiment. As an option, the user interfaces 500 may be implemented in the context of any one or more of the embodiments set forth in any previous and/or subsequent figure(s) and/or description thereof. Of course, however, the user interfaces 500 may be implemented in the context of any desired environment. Further, the aforementioned definitions may equally apply to the description below.

As shown, user interfaces 500 includes interface 502 which provides the patient with a notification that a prescription has been shipped. Next, in interface 504, a reminder or interaction is provided to the patient. For example, a reminder to take eye drops a set amount per day. Next, in interface 506, the system automatically determines when the prescription is running low, and automatically lines up a refill prescription to be sent.

In conventional systems, a patient would pick up a prescription, have to remember to take the prescription, and when the prescription runs out, would have to call the pharmacy to reorder the prescription, and then travel down to pick up the prescription. In the present system, the patient is automatically mailed the prescription, provided with reminders on when to take the prescription, and when it is automatically determined (based on rate of use of the prescription) that the prescription is running low, can automatically reorder and deliver the prescription. Thus, there are significant time savings in having a system where the patient does not have to manage the fulfillment of the order (i.e. the prescription), but can concentrate on simply taking the prescription and providing feedback to the medical provider as appropriate.

In one embodiment, an interface similar to interface 504 may request feedback from the patient (e.g. to determine if the prescription is working). Such feedback may be provided to the medical provider such that that automatic refill (of interface 506) may be adjusted based on such feedback.

Additionally, automatic flow and delivery of content may be based on a history of a patient. For example, if a first patient has a high rate of fidelity in taking a prescribed course of action, then the first patient may include a higher score associated with whether the prescription should be refilled at a predetermined time (when the prescription is due to run out). A second patient, however, may have a low rate of fidelity in taking the prescribed course of action, such that the second patient may have a lower score associated with whether the prescription should be refilled at a predetermined time. As such, before automatically refilling a prescription for the second patient, an additional interface may be sent requesting input on whether the second patient has taken the prescription as recommended, or input on whether the prescription is starting to run out. In this manner, metrics associated with a patient may dictate and control to what extent the system can operate in a more automatic manner.

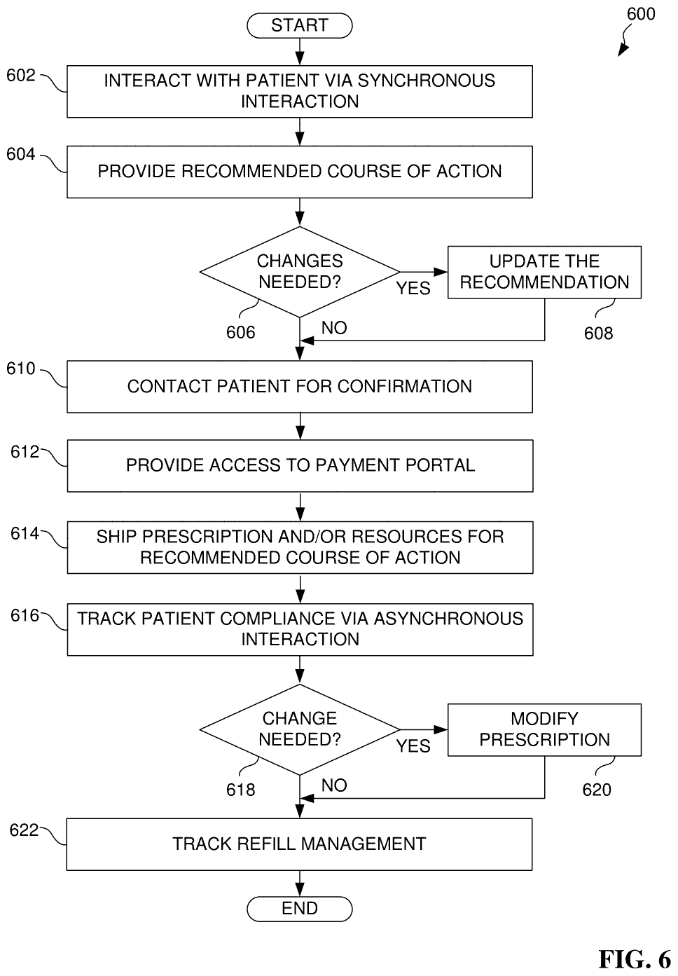

FIG. 6 illustrates a method 600 for tracking patient compliance and refill management, in accordance with one embodiment. As an option, the method 600 may be implemented in the context of any one or more of the embodiments set forth in any previous and/or subsequent figure(s) and/or description thereof. Of course, however, the method 600 may be implemented in the context of any desired environment. Further, the aforementioned definitions may equally apply to the description below.

As shown, the method 600 provides an exemplary scenario involving both synchronous and asynchronous aspects. The method 600 begins with interacting with a patient via a synchronous interaction. See operation 602. For example, the synchronous interaction may be any type of real-time event with a medical provider (such as but not limited to an in-person visit, a telehealth appointment, etc.). In response to the synchronous interaction, a recommended course of action is provided. See operation 604. In various embodiments, the recommended course of action may include a prescription, a follow up visit, a treatment, etc. As described hereinabove (see, e.g., operation 110), the recommendation (of operation 604) may be based on both the synchronous interaction as well as on prior asynchronous medical interactions.

It is determined whether any changes to the recommendation is needed. See decision 606. For example, after receiving a recommended course of action, a medical provider may provide input that the recommended prescription is out of budget for the patient, or the medical provider may be informed that the recommended prescription is more than 30 minutes away from where the patient lives. As such, the medical provider may modify and update the recommendation if needed. See operation 608.

After the synchronous interaction has ended, the patient may then be contacted for confirmation of the recommended course of action. See operation 610. For example, the confirmation may include any of a text message, an email, etc.

A patient may then be provided access to a payment portal. See operation 612. For example, the recommended course of action of operation 604 may include a prescription. The payment portal of operation 612 may be provided so that the patient can pay for the prescription given in the recommended course of action. Of course, the payment portal may be used for any purpose as it relates to the recommended course of action. In another embodiment, the payment portal may relate to the synchronous interaction itself (such as payment for the synchronous interaction).

The prescription and/or resources for the recommended course of action may then be shipped. See operation 614. In an alternative embodiment, a patient may desire immediate access to a prescription, and it may be indicated which pharmacy the patient can visit immediately to pick up the already paid-for prescription. In this manner, the method manages delivery of the prescription in any manner.

The patient compliance is then tracked via asynchronous interaction. See operation 616. For example, a tracking receipt (of the prescription being delivered) may be received, a communication from the patient may indicate that the medicine is working, etc. The patient compliance ultimately determines whether the patient is taking a course of action in compliance with the recommended course of action of operation 604. In view of such, the patient compliance of operation 616 may include any input associated with such compliance, including but not limited to feedback from smart devices, confirmation by automatic messaging service, response to SMS or email, feedback from smart pill bottle, and/or any other confirmation or feedback provided by the patient.

It is determined whether a change is needed. See decision 618. For example, based on the patient compliance of operation 616, it may be necessary to modify the prescription, the date to refill the prescription, the manner of usage, etc. As such, the prescription may be modified. See operation 620. Of course, it is to be appreciated that any change to the recommended course of action may be applied.

Further, refill management may be tracked. See operation 622. For example, if no change to the prescription is needed (per decision 618), then the prescription may be refilled (based on a predetermined schedule of rate of usage) automatically, and as displayed in the user interfaces 500, the prescription may be automatically refilled and sent out. If the prescription needs to be modified in any manner, such modification is applied and the prescription is then refilled.

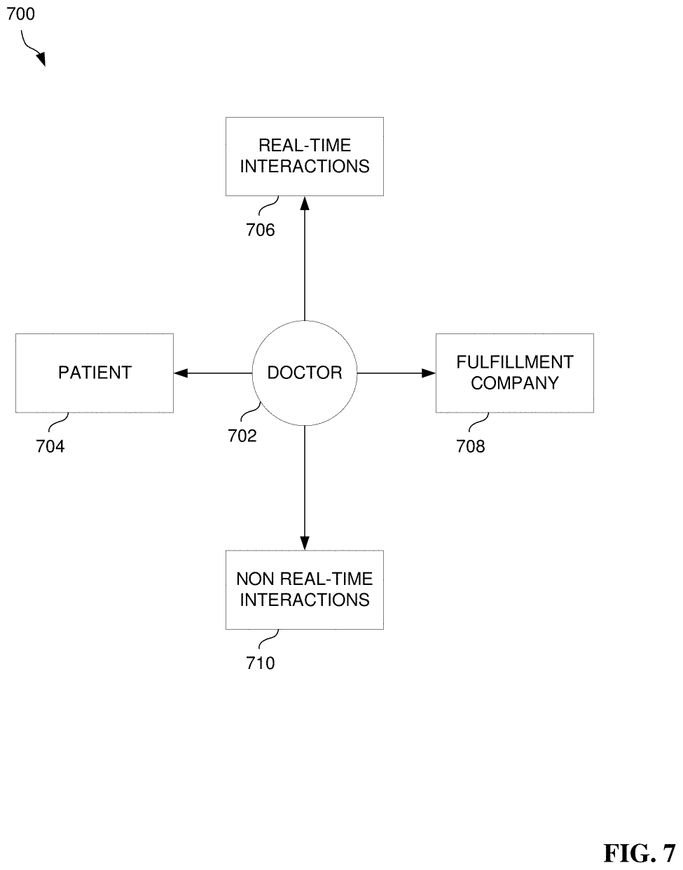

FIG. 7 illustrates a system 700 for doctor-led interaction, in accordance with one embodiment. As an option, the system 700 may be implemented in the context of any one or more of the embodiments set forth in any previous and/or subsequent figure(s) and/or description thereof. Of course, however, the system 700 may be implemented in the context of any desired environment. Further, the aforementioned definitions may equally apply to the description below.

As shown, the system 700 includes a doctor 702 who may interact with a patient 704 and with a fulfillment company 708. The doctor 702 may also engage in real-time interactions 706 (synchronous interactions) and with non real-time interactions 710 (i.e. asynchronous interactions). In one embodiment, the doctor-led interaction may include an omni-channel experience that allows the doctor 702, the patient 704 and the fulfillment company 708 to personalize each experience and compliance. Additionally, the doctor-led interaction may occur in office, in box, and at home with email, text, chat, phone, video, etc. As such, the doctor-led interaction may allow the doctor to more accurately predict the patient need(s) (based on additional data feedback and interaction) and modify a course of action to the patient 704 based on interactions (such as the real-time interactions 706 and/or the non real-time interactions 710).

Additionally, the fulfillment company 708 may be any resource prescribed to the patient 704 in relation to a recommended course of action. For example, the fulfillment company 708 may include a pharmacy, a physical therapy provider, post-discharge follow-up provider, a hospital, a telehealth provider, and/or any other resource that may enable the patient 704 to fulfill a recommended course of action from the doctor 702.

In one embodiment, the patient 704 may reach out to the fulfillment company 708 (such as a pharmacy) for assistance in diagnosing an issue based on symptoms. This may lead to an asynchronous interaction with the doctor 702 (referred to by the fulfillment company 708), whereupon the doctor 702 can provide recommendations to the patient 704 (e.g. via SMS messaging, via email, etc.). The patient 704 may provide feedback to the doctor 702. Eventually this interaction may lead, if needed, to a synchronous interaction (i.e. live exam) with the doctor 702 to assist the patient 704 in their recovery. The doctor 702 may use the fulfillment company to assist the patient 704 with any resources needed for their recovery.

In another embodiment, interaction between the doctor 702, the fulfillment company 708, and the patient 704 may be based on a subscription or membership arrangement. For example, the patient 704 may pay a monthly amount for medical interactions, which may give the patient 704 access to the doctor 702 and the fulfillment company 708 as needed. Such a membership may include initial diagnosis services, ongoing care services, asynchronous interactions, as well as synchronous interactions.

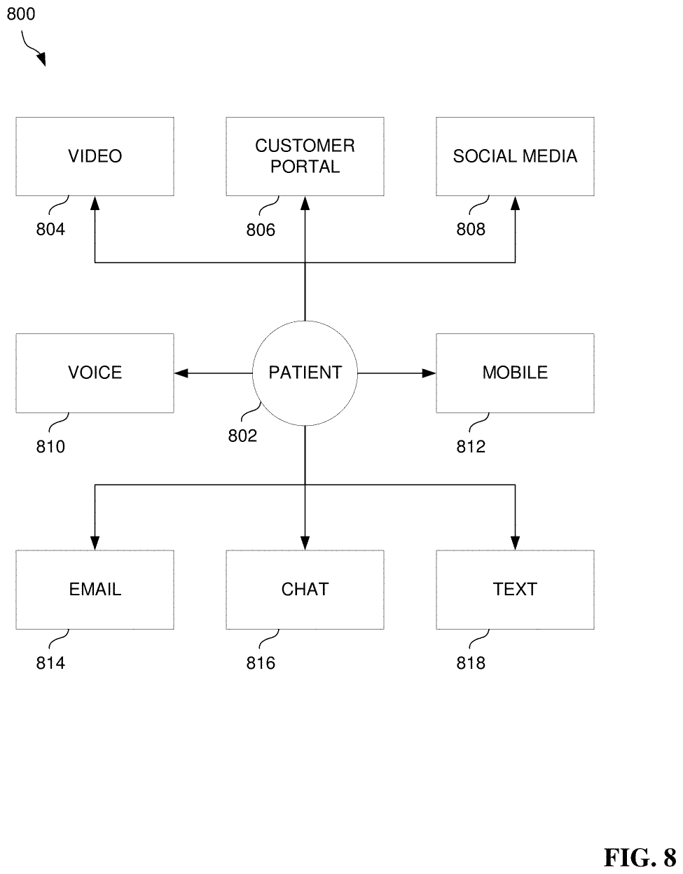

FIG. 8 illustrates a system 800 for patient-led communication, in accordance with one embodiment. As an option, the system 800 may be implemented in the context of any one or more of the embodiments set forth in any previous and/or subsequent figure(s) and/or description thereof. Of course, however, the system 800 may be implemented in the context of any desired environment. Further, the aforementioned definitions may equally apply to the description below.

As shown, the system 800 includes a patient 802 at the center of all types of communication, including video 804, customer portal 806, social media 808, mobile 812, text 818, chat 816, email 814, and/or voice 810. In various embodiments, the customer portal 806 may include a doctor or clinic web site or user interface for interacting with a patient.

In one embodiment, the patient 802 remains in control of all forms of communication. Additionally, data may be associated with each of video 804, customer portal 806, social media 808, mobile 812, text 818, chat 816, email 814, and voice 810. Such data may be additionally controlled (including dissemination of the data back to the medical provider) by the patient 802.

Additionally, data from a first source of communication may lead to data obtained from a second source of communication. For example, a telehealth communication (via, e.g., a video 804) may include information on a recommended course of action, which in turn may be followed up with a reminder by email of the recommended course of action, and a subsequent follow up on whether the course of action was completed. Regardless of the form of communication, the patient 802 may remain in control of the data from all communication sources.

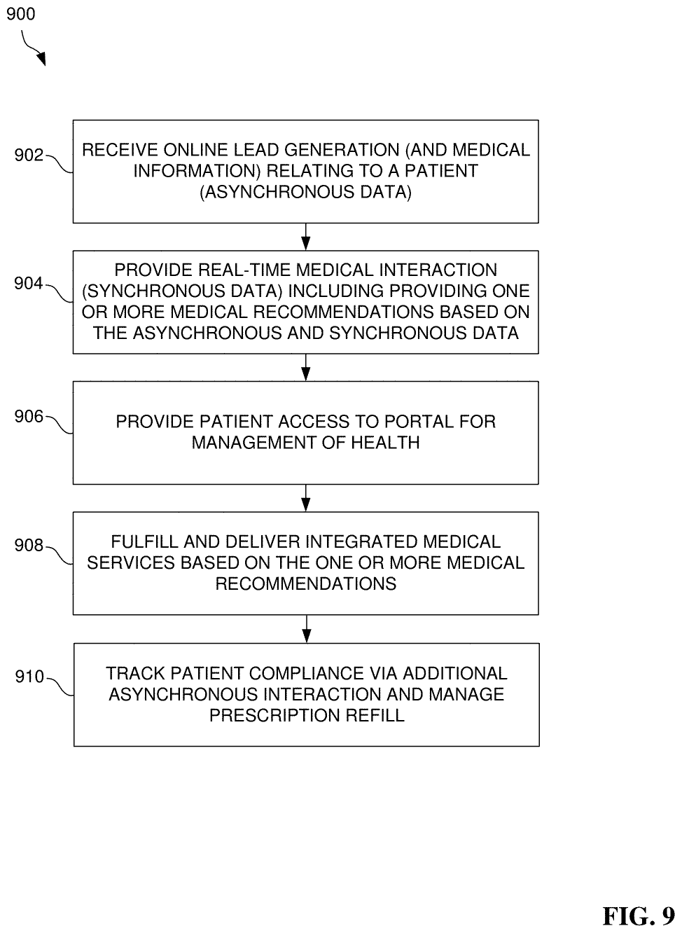

FIG. 9 illustrates a method 900 for providing an omni-channel support and medical delivery system, in accordance with one embodiment. As an option, the method 900 may be implemented in the context of any one or more of the embodiments set forth in any previous and/or subsequent figure(s) and/or description thereof. Of course, however, the method 900 may be implemented in the context of any desired environment. Further, the aforementioned definitions may equally apply to the description below.

As shown, the method 900 starts by receiving online lead generation (and medical information) relating to a patient. See operation 902. Such online lead generation may be provided by another medical care provider, a pharmacy, etc. The data associated with the online lead generation and/or the medical information may comprise asynchronous data.

Real-time medical interaction (synchronous data) may be provided including providing one or more medical recommendations based on the asynchronous and synchronous data. See operation 904. In response to the medical recommendations, the patient is provided access to a portal for management of health. See operation 906. For example, a medical recommendation may include lowering blood sugar levels. The portal for management of health may include a field for inputting daily a blood sugar level. The portal of management of health may further send reminders to the patient to test their blood sugar level.

Next, integrated medical services are fulfilled and delivered based on the one or more medical recommendations. See operation 908. For example, a prescription relating to a medical recommendation may be fulfilled and delivered to the patient.

Patient compliance is tracked via additional asynchronous interaction and/or prescription refill is managed. See operation 910. For example, any feedback the patient provides in relation to the medical recommendation may be tracked (compliant with patient defined privacy settings). Additionally, as discussed herein, the prescription refill may be automatically managed (see, e.g., user interface 506). The additional asynchronous interaction may include any further non real-time interaction by the patient after receipt of the one or more medical recommendations.

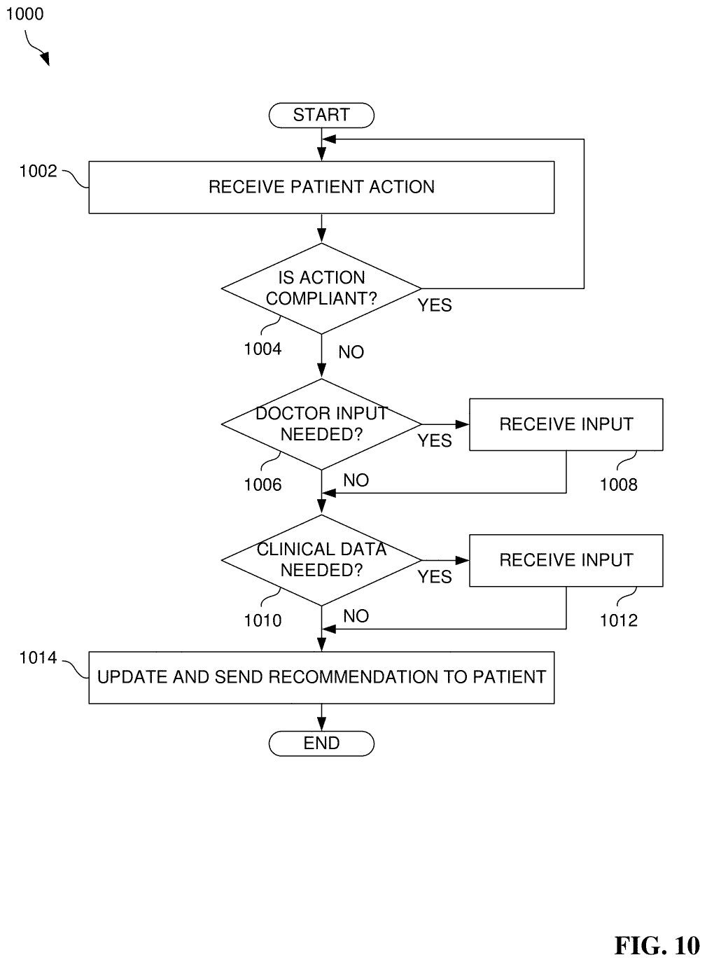

FIG. 10 illustrates a method 1000 for updating and sending recommendations to patient, in accordance with one embodiment. As an option, the method/system 1000 may be implemented in the context of any one or more of the embodiments set forth in any previous and/or subsequent figure(s) and/or description thereof. Of course, however, the method/system 1000 may be implemented in the context of any desired environment. Further, the aforementioned definitions may equally apply to the description below.

As shown, the method 1000 starts by receiving patient action. See operation 1002. The patient action may include action associated with a medical recommendation. Next, it is determined whether the action is compliant. See decision 1004. If the action is compliant with the medical recommendation, then no further action is taken. If the action is not compliant with the medical recommendation, it is determined whether doctor input is needed. See decision 1006. If doctor input is needed, input is received from the doctor. See operation 1008. For example, if the patient failed to take all indicated doses of a medical drug, the doctor may decide to extend the number of days of taking the medical drug. In another embodiment, if the patient experienced an allergic reaction in response to taking a recommended medicine, the doctor may change the medicine.

It is determined whether clinical data is needed. See decision 1010. For example, clinical studies may document a reaction to a medicine with possible alternatives to such medicine. Additionally, there may be case studies where a patient that failed to complete the expected length of medicine (in terms of days) may need an unobvious next course of action to rectify the deficiency. If clinical data is needed, the doctor may choose to receive such input. See operation 1012.

Lastly, the recommendation is updated and sent to the patient. See operation 1014. Such updated recommendation may be based on the additional asynchronous data, including both or one of the patient action and clinic data sets.

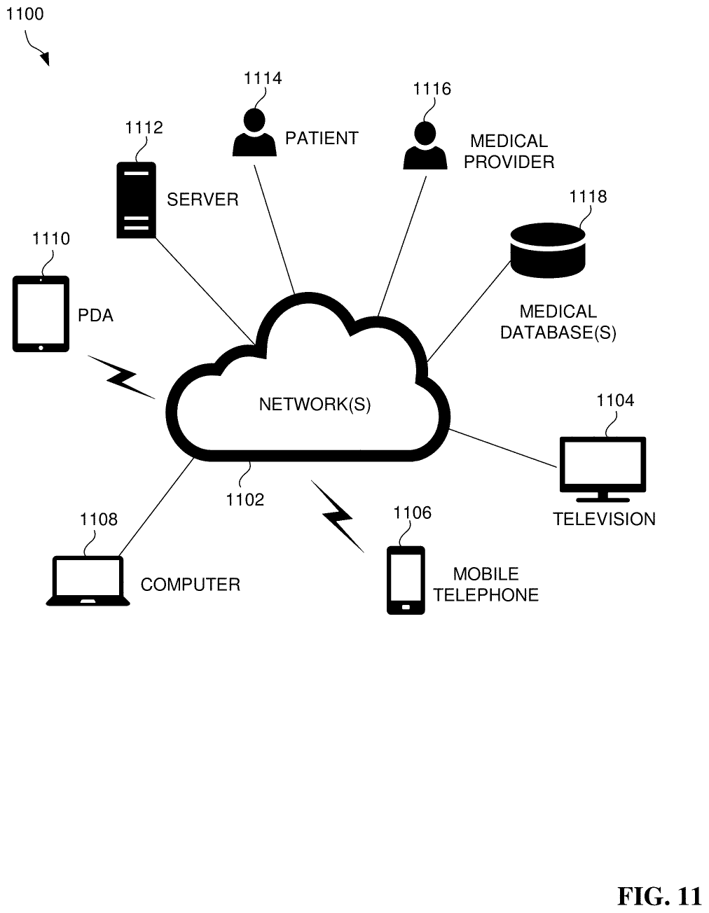

FIG. 11 illustrates a network architecture 1100, in accordance with one possible embodiment. As shown, at least one network 1102 is provided. In the context of the present network architecture 1100, the network 1102 may take any form including, but not limited to a telecommunications network, a local area network (LAN), a wireless network, a wide area network (WAN) such as the Internet, peer-to-peer network, cable network, etc. While only one network is shown, it should be understood that two or more similar or different networks 1102 may be provided.

Coupled to the network 1102 is a plurality of devices. For example, a server computer 1112 and an end user computer 1108 may be coupled to the network 1102 for communication purposes. Such end user computer 1108 may include a desktop computer, lap-top computer, and/or any other type of logic. Still yet, various other devices may be coupled to the network 1102 including a personal digital assistant (PDA) device 1110, a mobile phone device 1106, a television 1104, etc. Additionally, medical related database(s) 1118 may be coupled to the network 1102.

Further, coupled to the network 1102 may include any number of entities, including but not limited to, patient 1114, and medical provider 1116. Although not shown, it is to be understood that any entity and number of entities may be connected to the network 1102. For example, a pharmacy provider, an in-home provider, a telehealth provider, may in like manner be connected to the network 1102.

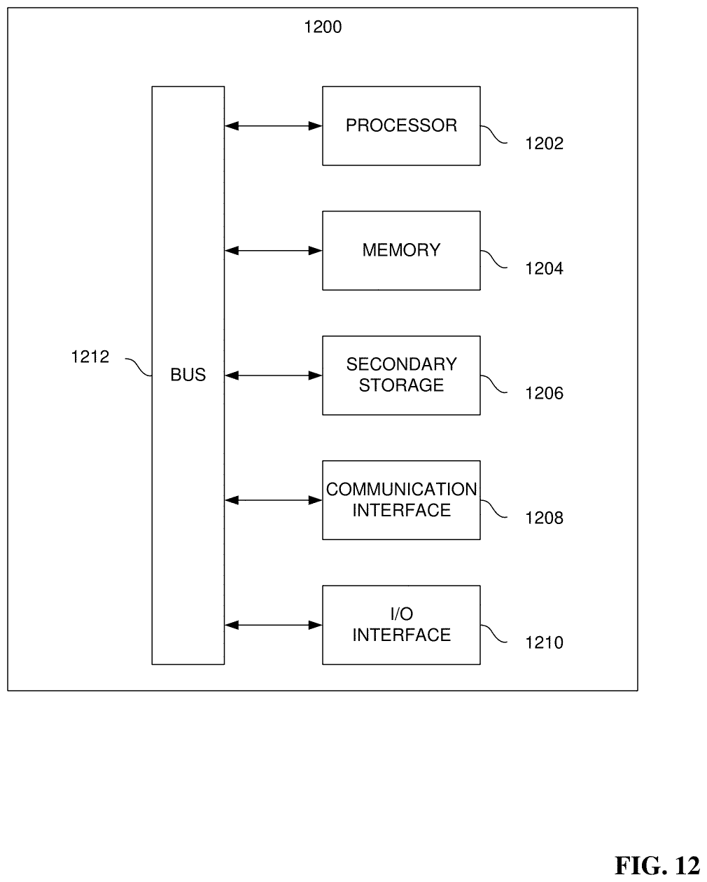

FIG. 12 illustrates an exemplary system 1200, in accordance with one embodiment. As an option, the system 1200 may be implemented in the context of any of the devices of the network architecture 1100 of FIG. 11. Of course, the system 1200 may be implemented in any desired environment.

As shown, a system 1200 is provided including at least one central processor 1202 which is connected to a communication bus 1212. The system 1200 also includes main memory 1204 [e.g. random access memory (RAM), etc.]. The system 1200 also includes a graphics processor 1208 and a display 1210.

The system 1200 may also include a secondary storage 1206. The secondary storage 1206 includes, for example, a hard disk drive and/or a removable storage drive, representing a floppy disk drive, a magnetic tape drive, a compact disk drive, etc. The removable storage drive reads from and/or writes to a removable storage unit in a well known manner.

Computer programs, or computer control logic algorithms, may be stored in the main memory 1204, the secondary storage 1206, and/or any other memory, for that matter. Such computer programs, when executed, enable the system 1200 to perform various functions (as set forth above, for example). Memory 1204, storage 1206 and/or any other storage are possible examples of non-transitory computer-readable media. It is noted that the techniques described herein, in an aspect, are embodied in executable instructions stored in a computer readable medium for use by or in connection with an instruction execution machine, apparatus, or device, such as a computer-based or processor-containing machine, apparatus, or device. It will be appreciated by those skilled in the art that for some embodiments, other types of computer readable media are included which may store data that is accessible by a computer, such as magnetic cassettes, flash memory cards, digital video disks, Bernoulli cartridges, random access memory (RAM), read-only memory (ROM), and the like.

As used here, a “computer-readable medium” includes one or more of any suitable media for storing the executable instructions of a computer program such that the instruction execution machine, system, apparatus, or device may read (or fetch) the instructions from the computer readable medium and execute the instructions for carrying out the described methods. Suitable storage formats include one or more of an electronic, magnetic, optical, and electromagnetic format. A non-exhaustive list of conventional exemplary computer readable medium includes: a portable computer diskette; a RAM; a ROM; an erasable programmable read only memory (EPROM or flash memory); optical storage devices, including a portable compact disc (CD), a portable digital video disc (DVD), a high definition DVD (HD-DVD™), a BLU-RAY disc; and the like.

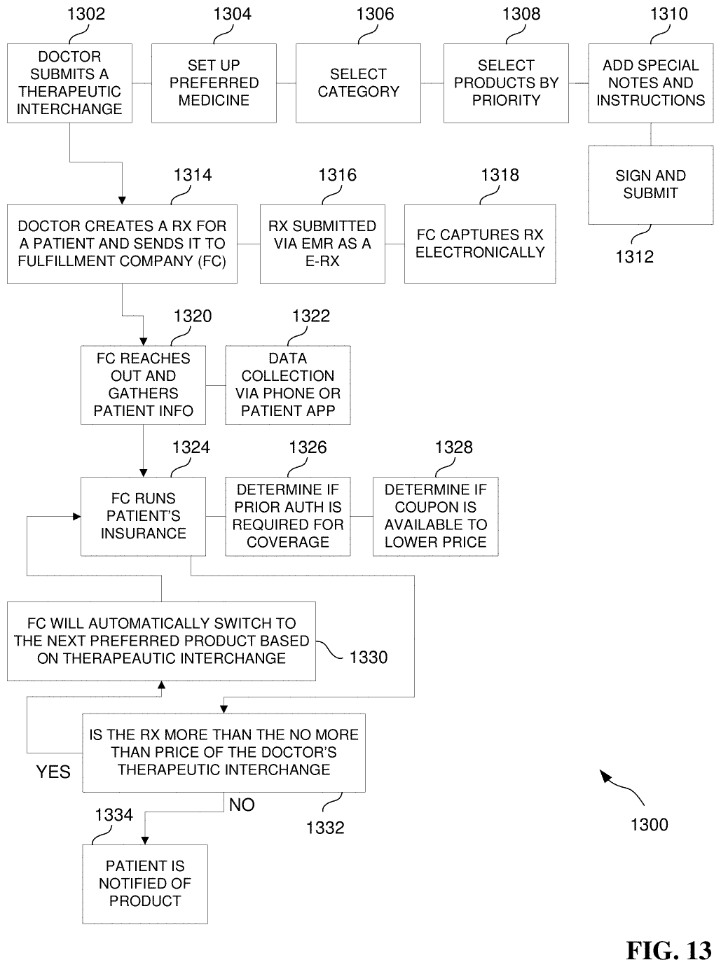

FIG. 13 illustrates an exemplary therapeutic interchange 1300, in accordance with one embodiment. As an option, the exemplary therapeutic interchange 1300 may be implemented in the context of any one or more of the embodiments set forth in any previous and/or subsequent figure(s) and/or description thereof. Of course, however, the exemplary therapeutic interchange 1300 may be implemented in the context of any desired environment. Further, the aforementioned definitions may equally apply to the description below.

As shown, the exemplary therapeutic interchange 1300 starts with operation 1302 where a doctor submits a therapeutic interchange. Steps and details relating to operation 1302 may include operations 1304-1312. At operation 1304, preferred medication may be inputted. At operation 1306 a category (or more than one) of the medicine may be selected. At operation 1308, a product(s) (of medicine) may be selected by priority. Further, at operation 1310, special note(s) and/or instruction(s) (from the doctor) may be added. At operation 1312, the doctor may sign and submit the preferred medication. In this manner, doctors may select a preferred medication.

The doctor, per operation 1314, may then create a prescription (RX) for a patient and send it to a fulfillment company (FC). Steps and details relating to operation 1314 may include operations 1316-1318. Per operation 1316, the RX may be submitted via an electronic medical record (EMR) as an electronic prescription (e-RX). Per operation 1318, the FC may capture and/or otherwise receive the RX electronically.

The FC may reach out, per operation 1320, and gather patient info. In this manner, the FC may proactively reach out to the patient to take action. A step and detail relating to operation 1320 may include operation 1322. Per operation 1322, data collection may occur via a phone, a patient app, and/or any other portal (e.g. browser, etc.).

The FC, per operation 1324 may then run the patient's insurance. Steps and details relating to operation 1324 may include operations 1326-1328. Per operation 1326, it is determined if prior authorization is required for coverage. In one embodiment, if prior authorization is required, it is requested. Per operation 1328, it is determined if a coupon is available to lower price. In one embodiment, if a coupon is available, it is applied.

It is then determined, per operation 1332, whether the RX is more than the no more than price of the doctor's therapeutic interchange. In one embodiment, the no more than price may be a pre-determined maximum price indicated by the patient, or a maximum price that the doctor does not want their patient to pay. If the RX is more than the no more price, the interchange continues on to operation 1330 where the FC will then automatically switch to the next preferred product based on the therapeutic interchange.

Once the RX is less than the no more than price, the interchange continues on to operation 1334 where the patient is notified of the product.

In one embodiment, the therapeutic interchange 1300 may follow after a doctor conducts and provides a diagnosis. The doctor may then determine a proper treatment for the diagnosis. As part of the proper treatment, the therapeutic interchange may be used to determine a relevant product which conforms with input(s) by the doctor as well as input(s) by the patient (e.g. including patient attributes, etc.). In this manner, the product selected and presented (per operation 1334) may be dictated by the doctor, but also take into consideration personal attributes and preferences from a patient (e.g. location, price constraint, allergies, lifestyle, etc.).

In other embodiments, the therapeutic interchange 1300 may also allow doctors to rank substitute medications following their preferred medication (per operations 1304-1312). Further, a doctor may authorize the FC to switch the product selected (via operations 1324-1334) based on preferences (of both the doctor and the patient) based on a predetermined ranked order. For example, the doctor may indicate that price is a higher priority over location. The patient may indicate that they have an allergy (e.g. egg, etc.) which may have a higher priority over generic versus brand name. The doctor may receive preferences from the patient and control the overall ranked order of the available preferences.

Additionally, it should be noted that the therapeutic interchange 1300 focuses primarily on the flow from operation 1302 to operation 1314 to operation 1320 to operation 1324 to decision 1332 (which may involve operation 1330) to operation 1334. To that end, the therapeutic interchange 1300 includes an arrow from one operation to the next. In contrast, the sub elements under each operation are intended as details associated with each operation/decision of the flow. Thus, for example, operations 1304-1312 pertain to details of operation 1302, operations 1316-138 pertain to details of operation 1314, etc.

It should be understood that the arrangement of components illustrated in the Figures described are exemplary and that other arrangements are possible. It should also be understood that the various system components (and means) defined by the claims, described below, and illustrated in the various block diagrams represent logical components in some systems configured according to the subject matter disclosed herein.

For example, one or more of these system components (and means) may be realized, in whole or in part, by at least some of the components illustrated in the arrangements illustrated in the described Figures. In addition, while at least one of these components are implemented at least partially as an electronic hardware component, and therefore constitutes a machine, the other components may be implemented in software that when included in an execution environment constitutes a machine, hardware, or a combination of software and hardware.

More particularly, at least one component defined by the claims is implemented at least partially as an electronic hardware component, such as an instruction execution machine (e.g., a processor-based or processor-containing machine) and/or as specialized circuits or circuitry (e.g., discreet logic gates interconnected to perform a specialized function). Other components may be implemented in software, hardware, or a combination of software and hardware. Moreover, some or all of these other components may be combined, some may be omitted altogether, and additional components may be added while still achieving the functionality described herein. Thus, the subject matter described herein may be embodied in many different variations, and all such variations are contemplated to be within the scope of what is claimed.

In the description above, the subject matter is described with reference to acts and symbolic representations of operations that are performed by one or more devices, unless indicated otherwise. As such, it will be understood that such acts and operations, which are at times referred to as being computer-executed, include the manipulation by the processor of data in a structured form. This manipulation transforms the data or maintains it at locations in the memory system of the computer, which reconfigures or otherwise alters the operation of the device in a manner well understood by those skilled in the art. The data is maintained at physical locations of the memory as data structures that have particular properties defined by the format of the data. However, while the subject matter is being described in the foregoing context, it is not meant to be limiting as those of skill in the art will appreciate that various of the acts and operations described hereinafter may also be implemented in hardware.

To facilitate an understanding of the subject matter described herein, many aspects are described in terms of sequences of actions. At least one of these aspects defined by the claims is performed by an electronic hardware component. For example, it will be recognized that the various actions may be performed by specialized circuits or circuitry, by program instructions being executed by one or more processors, or by a combination of both. The description herein of any sequence of actions is not intended to imply that the specific order described for performing that sequence must be followed.

All methods described herein may be performed in any suitable order unless otherwise indicated herein or otherwise clearly contradicted by context.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

The embodiments described herein included the one or more modes known to the inventor for carrying out the claimed subject matter. Of course, variations of those embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

What is claimed is:

1. A non-transitory computer readable storage medium storing one or more programs, the one or more programs comprising instructions which, when executed by an apparatus, cause the apparatus to:
store asynchronous medical data associated with a patient;
store synchronous medical data associated with the patient;
integrate the asynchronous medical data and the synchronous medical data to create cohesive patient data;
extract relevant data from clinical databases based on the cohesive patient data;
provide one or more decisions based on the cohesive patient data and the relevant data.

2. The non-transitory computer readable storage medium of claim 1, wherein the asynchronous medical data includes non real-time data.

3. The non-transitory computer readable storage medium of claim 2, wherein the non real-time data includes at least one of:
patient medical history;
patient and medical provider interaction;
patient medical procedure;
patient medical condition;
patient medical allergy;
family medical procedure;
family medical condition;
family medical allergy;
family health history;
prior medical interaction;
prior medical diagnosis;
prior prescription;
prior illness;
prior surgery;
prior immunization;
results of physical exams;
results of genetic test;
frequency of past screening,
substance abuse;
pregnancy complications;
prior hospitalization;
communication associated with the patient;
lifestyle habits;
personal data associated with the patient; or
remote patient monitoring.

4. The non-transitory computer readable storage medium of claim 2, wherein the non real-time data includes a patient action which includes at least one of receipt of medical treatment, refill of a prescription, decision to not take medical action, refusal of medical treatment, election of an alternative treatment, selection of a lower-cost option, response to or sending a communication associated with a medical representative, change a health insurance plan, fulfillment of medical prescription, completion of medical prescription, tracking of dosage, request for refill, indication of drug side effect, completion of daily reminder, completion of interaction, tracking of exercise, tracking of diet, tracking of food intake, body vital measurements, body vital statistics, body vital historical data, or fulfillment of medical prescription coupon.

5. The non-transitory computer readable storage medium of claim 1, wherein the synchronous medical data include real-time data.

6. The non-transitory computer readable storage medium of claim 5, wherein the real-time data includes at least one of:
real-time behavior observations;
real-time communication;
digital communication;
video patient evaluations;
health triage;
in-person visit;
live examination;
telehealth communication;
in-house provider visit;
in-office provider visit; or
current vitals of the patient.

7. The non-transitory computer readable storage medium of claim 1, wherein the synchronous medical data is received in-person.

8. The non-transitory computer readable storage medium of claim 1, wherein the synchronous medical data is received digitally.

9. The non-transitory computer readable storage medium of claim 1, wherein the one or more programs comprises instructions which, when executed by the apparatus, cause the apparatus to store the asynchronous medical data on a remote server, wherein the remote server is at least one of: a cloud based server, an online electronic medical record system, or online data storage.

10. The non-transitory computer readable storage medium of claim 9, wherein the remote server is HIPAA-compliant.

11. The non-transitory computer readable storage medium of claim 1, wherein the one or more programs comprises instructions which, when executed by the apparatus, cause the apparatus to anonymize the cohesive patient data prior to extracting the relevant data.

12. The non-transitory computer readable storage medium of claim 1, wherein the one or more programs comprises instructions which, when executed by the apparatus, cause the apparatus to receive additional input from a physician based on the one or more decisions.

13. The non-transitory computer readable storage medium of claim 12, wherein the additional input includes at least one of pricing considerations, lifestyle considerations, availability considerations.

14. The non-transitory computer readable storage medium of claim 1, wherein the asynchronous medical data and the synchronous medical data each include privacy and personal security settings set by the patient.

15. The non-transitory computer readable storage medium of claim 1, wherein the one or more programs comprises instructions which, when executed by the apparatus, cause the apparatus to solicit feedback from the patient in relation to the one or more decisions.

16. The non-transitory computer readable storage medium of claim 15, wherein the one or more programs comprises instructions which, when executed by the apparatus, cause the apparatus to provide to the patient an incentive associated with the feedback.

17. The non-transitory computer readable storage medium of claim 15, wherein the feedback is used as additional asynchronous medical data.

18. The non-transitory computer readable storage medium of claim 17, wherein the one or more programs comprises instructions which, when executed by the apparatus, cause the apparatus to update the one or more decisions based on the additional asynchronous medical data.

19. A computer-implemented method, comprising:
storing, using a processor, asynchronous medical data associated with a patient;
storing, using the processor, synchronous medical data associated with the patient;
integrating, using the processor, the asynchronous medical data and the synchronous medical data to create cohesive patient data;
extracting, using the processor, relevant data from clinical databases based on the cohesive patient data;
providing, using the processor, one or more decisions based on the cohesive patient and the relevant data.

20. A device, comprising:
a non-transitory memory storing instructions; and
one or more processors in communication with the non-transitory memory, wherein the one or more processors execute the instructions to:
store asynchronous medical data associated with a patient;
store synchronous medical data associated with the patient;
integrate the asynchronous medical data and the synchronous medical data to create cohesive patient data;
extract relevant data from clinical databases based on the cohesive patient data;
provide one or more decisions based on the cohesive patient and the relevant data.
